SEQUENCE LISTING (10 sequences)

SEQ ID NO: 1 - protein, 10 aa, Artificial Sequence
  Description of Artificial Sequence Synthetic peptide
HTKGFAATET

SEQ ID NO: 2 - protein, 5 aa, Artificial Sequence
  Description of Artificial Sequence Synthetic peptide
AGGVA

SEQ ID NO: 3 - protein, 8 aa, Artificial Sequence
  Description of Artificial Sequence Synthetic peptide
FGSPLITN

SEQ ID NO: 4 - protein, 4 aa, Artificial Sequence
  Description of Artificial Sequence Synthetic peptide
KLQE

SEQ ID NO: 5 - protein, 10 aa, Artificial Sequence
  Description of Artificial Sequence Synthetic peptide
SVASLILTTE

SEQ ID NO: 6 - protein, 10 aa, Artificial Sequence
  Description of Artificial Sequence Synthetic peptide
VTRSALQNAG

SEQ ID NO: 7 - protein, 26 aa, Artificial Sequence
  Description of Artificial Sequence Synthetic peptide
TYRVIERVSGYAPEYVSFVNGVVTIK

SEQ ID NO: 8 - protein, 164 aa, Artificial Sequence
  Description of Artificial Sequence Synthetic polypeptide
TYRVIERVSGYAPEYVSFVNGVVTIKGGTYRVIERVSGYAPEYVSFVNGVVTIKGGHTKGFAATETGAGGVAGFGSPLITNGKLQEGSVASLILTTEGVTRSALQNAGGGTYRVIERVSGYAPEYVSFVNGVVTIKGGTYRVIERVSGYAPEYVSFVNGVVTIK

SEQ ID NO: 9 - protein, 239 aa, Artificial Sequence
  Description of Artificial Sequence Synthetic polypeptide
DQKSHTATSMKIETPATNAAGQTTATVDLKTNQVSVADQKVSLNTISEGMTPEAATTIVSPMKTYSSAPALKSKEVLAQEQAVSQAAANEQVSPAPVKSITSEVPAAKAVAQPASTTNAVAAHPENAGLQPHVAAYKEKVASTYGVNEFSTYRAGDPGDHGKGLAVDFIVGTNQALGNKVAQYSTQNMAANNISYVIWQQKFYSNTNSIYGPANTWNAMPDRGGVTANHYDHVHVSFNK

SEQ ID NO: 10 - protein, 501 aa, Artificial Sequence
  Description of Artificial Sequence Synthetic polypeptide
DQKSHTATSMKIETPATNAAGQTTATVDLKTNQVSVADQKVSLNTISEGMTPEAATTIVSPMKTYSSAPALKSKEVLAQEQAVSQAAANEQVSPAPVKSITSEVPAAKEEVKPTQTSVSQSTTVSPASVAAETPAPVAKVAPVRTVAAPRVASVKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGTNQALGNKVAQYSTQNMAANNISYVIWQQKFYSNTNSIYGPANTWNAMPDRGGVTANHYDHVHVSFNKTYRVIERVSGYAPEYVSFVNGVVTIKGGTYRVIERVSGYAPEYVSFVNGVVTIKGGHTKGFAATETGAGGVAGFGSPLITNGKLQEGSVASLILTTEGVTRSALQNAGGGTYRVIERVSGYAPEYVSFVNGVVTIKGGTYRVIERVSGYAPEYVSFVNGVVTIK

(19) United States
(12) Patent Application Publication
(10) Pub. No.: US 2024/0010690 A1
(43) Pub. Date: Jan. 11, 2024

(54) ORAL VACCINE, METHOD OF PREPARATION AND USE THEREOF

(71) Applicant: FeedVax, Inc., Honolulu, HI (US)

(72) Inventor: Luis BARLETTA, Honolulu, HI (US)

(21) Appl. No.: 18/255,828

(22) PCT Filed: Dec. 2, 2021
(86) PCT No.: PCT/US2021/061656

§ 371 Date: Jun. 2, 2023

Related U.S. Application Data
(60) Provisional application No. 63/120,511, filed on Dec. 2, 2020.

(51) Int. Cl. C07K 14/315; A61K 9/00; A61K 31/716; A61K 36/889; A61K 47/38; A61P 37/04; C12N 15/70; A61P 31/04

(52) U.S. Cl. CPC C07K 14/315; A61K 9/0056; A61K 31/716; A61K 36/889; A61K 47/38; A61P 37/04; C12N 15/70; A61P 31/04

(57) ABSTRACT

The present invention provides a composition comprising a chimeric fusion protein and immunmodulators that are supported on a carrier together with a solution as a vehicle. In other aspects the invention is related to associated polynucleotides, chimeric peptides, and methods for the preparation of the composition and for the prevention of bacterial infections in fish by administration of an oral vaccine form.

### CROSS-REFERENCE TO RELATED APPLICATIONS

This application claims the benefit of priority to U.S. provisional patent application Ser. No. 63/120,511, filed Dec. 2, 2020, the contents of which are incorporated herein by reference and made a part hereof.

### SEQUENCE LISTING

The present application contains a Sequence Listing that has been submitted electronically in ASCII format and is hereby incorporated by reference in its entirety. The ASCII copy, created on Nov. 15, 2021, is named AQUA FISH .txt and is 10 KB in size.

### FIELD OF INVENTION

This invention is related to vaccines against infectious agents, method of preparation and use thereof as a vaccine for oral administration in fish.

### BACKGROUND OF THE INVENTION

In a population of fish in their natural environment, diseases have been considered a normal part of the biological process. In fish breeding this situation gradually changed due to the number and density of fish. Diseases considered a phenomenon in wild populations are sometimes a problem on a fish farm. During the short history of modern aquaculture it is accepted that disease prevention based on stimulation of the immune system has become an integral part of management operations in aquaculture (Fish Vaccination Edited by Roar Gudding, Atle Lillehaug and Oystein Evensen. 2014. John Wiley & Sons, Ltd.).

Chaperonins are oligomeric proteins that assist in the folding of nascent or denatured proteins. Bacterial chaperonins are strongly immunogenic and cause pathologies in different tissues. The group of bacterial chaperonins 60 are an important factor for the generation of the immune response against pathogens, activating the expression of pro-inflammatory cytokines (J C Ranford and B Henderson, Chaperonins in disease: mechanisms, models, and treatments, Mol Pathol. 2002 August; 55(4): 209-213, https://dx.doi.org/10.1136%2Fmp.55.4.209).

Within the group of gram positive bacteria, surface anchor proteins are involved both in adhesion events on host cells and in the mechanism of pathogenesis (Timothy J. Foster, The MSCRAMM Family of Cell-Wall-Anchored Surface Proteins of Gram-Positive Cocci, Trends in Microbiology, Volume 27, Issue 11, November 2019, Pages 927-941, https://doi.org/10.1016/j.tim.2019.06.007).

Multivalent and multiepitope vaccines combining at least three segments or epitopes conjugated by linkers have been presented as alternative strategies for the prevention and control of diseases (Nefasat, N. et al. Designing an efficient multi-epitope peptide vaccine against Vibrio cholera via combined immunoinformatics and protein interaction based approaches. Comput Biol Chem. 62, 82-95, 2016). In addition, bioinformatic approaches have been applied to design suitable multivalent and multiepitope vaccines (Hajighahramani, N. et al. Immunoinformatics analysis and in silico designing of a novel multi-epitope peptide vaccine against Staphylococcus aureus. Infect. Genet. Evol. 48, 83-94, 2017). Each individual epitope on a chimeric peptide can provide a highly effective vaccine by inducing and increasing a specific humoral response in addition to other cellular responses (Zhao, Z. et al. Multiple B-cell epitope vaccine induces a Staphylococcus enterotoxin B-specific lgG1 protective response against MRSA infection. Sci Rep. 5, 12371, https://doi.org/10.1038/srep12371, 2015). However, adequate linkers have been considered to minimize steric effects of each chimeric epitope and increase the presentation of said epitopes to the host immune system (Farhadi, T. et al. Designing of complex multi-epitope peptide vaccine based on Omps of Klebsiella pneumonia: an in silico approach. Int J Pept Res Ther. 21, 325-341, 2015).

Therefore, there is a need to develop vaccines to stimulate the immune system, starting with the interaction at the level of the digestive tract, for the prevention of bacterial infections in fish.

### BRIEF DESCRIPTION OF THE INVENTION

In a general embodiment, the invention provides a composition for an orally administered vaccine based on a chimeric fusion protein, which is supported on a carrier for being added to a food. The chimeric fragment of the chimeric fusion protein comprises epitopes of a HSP60 chaperonin and epitopes of a pili PI-2a anchor protein.

In an embodiment of the invention, a chimeric peptide named Q in the present application comprises from the N terminal end a residue sequence of amino acid type of at least two equal or different epitopes or domains or fragments in any order, presented by any from SEQ ID No 1 to SEQ ID No 7 or any of the homologous residue sequences of amino acid type from SEQ ID No 1 to SEQ ID No 7, wherein said epitopes are attached among them by an amino acid type linker which can preferably be one or two glycines, alanines or valines.

The sequences from SEQ ID No 1 to SEQ ID No 6, represent conserved domains of 60 KDa chaperonins found in bacteria of Streptococcus genus, Lactococcus y others. These sequences as information without being the only records that contain them are found by example in the GenBank with accession numbers WP_084786044, WP_161941966.1, WP_079474151.1, CAC7457577.1, WP_097025287.1 y WP_053348953.1, respectively.

The sequence SEQ ID No 7, represents a conserved domain from different bacteria including bacteria of the Streptococcaceae family, belonging to a pili anchor protein. This sequence as information without being the only record that contains it is found by example in the GenBank with accession number ACM07465.1.

In an additional embodiment of the invention it is provided a chimeric fusion protein comprising a region called Q (region representing an artificial chimeric peptide that enhances the immune response) attached to another region called F (region representing a fusion peptide or fusion protein), wherein the region F is in the N terminal end and it is a protein or fragment known for stimulating the immune system in fish.

In other embodiment of the invention, the region F of the chimeric fusion protein can be a protein or fragments thereof, homologous sequences of amino acid type or fragments thereof corresponding to a Modified Surface immunogenic protein (SIP) with sequence SEQ ID No 9. This sequence as information without being the only record that contains it is found by example in the GenBank with accession number AEK06226.1.

In other embodiment of the invention of the present application said chimeric fusion protein is part of a composition comprising immunomodulators such as palm oil and yeast β-glucan, supported on a carrier of chitosan and alginate

Likewise, in an embodiment of the invention, the composition is given to the fish orally using a solution of carboxymethylcellulose as vehicle for dosing alone or in a mixture with food, in order to avoid additional handling of fish that would lead to episodes of stress.

The use of the composition as vaccine is focused on the prevention of diseases caused by bacteria in fish breeding.

Also, other embodiment of the invention provides a method for preparation of the composition as oral vaccine for fish comprising
a) Find in databases epitopes of pathogens of teleost fish that have been described as important in the development of cellular and/or humoral immunity.
b) Join these sequences to obtain the chimeric peptide Q and/or fuse them with specific antigenic sequences to obtain the chimeric fusion protein FQ.
c) Perform the non-biological synthesis or biological synthesis of a chimeric peptide Q and/or a chimeric fusion protein FQ.
d) Purify the chimeric peptide Q and/or the chimeric fusion protein FQ.
e) Produce a composition mixing immunomodulators and generating the crosslinking or entrapment in the web or capsule considered as carrier in the presence of the chimeric peptide Q and/or the chimeric fusion protein FQ.
f) Mix the composition with a solution considered as vehicle for dosing and use as vaccine.

### BRIEF DESCRIPTION OF THE FIGURES

FIG. 1 shows an electrophoresis SDS-PAGE gel stained with Coomasie Blue wherein in lanes 2 and 3 appears a chimeric fusion protein FQ.

FIG. 2 shows a Western Blot (anti His) wherein in lanes 2 and 3 appears a chimeric fusion protein FQ.

FIG. 3 shows a graph with the kinetics of antibody titer of animals immunized with 200 μg of SIP-FUSION vs 200 μg of SIP.

FIG. 4 shows a graph with the survival percent from the challenge test against Streptococcus agalactiae (UEL12).

### DEFINITIONS

Antigen: Compound that upon entering the body induces an immune response, causing the formation of antibodies.

Immune response: Body's defense mechanism against compounds considered harmful or strange.

Digestive tract: a series of hollow organs that form a tube that runs from the mouth to the anus.

Epitope: or antigenic determinant is the portion of a macromolecule that is recognized by the immune system, specifically the sequence to which antibodies bind, which are receptors on B cells or T cells in a soluble state.

Domain: Amino acid sequence with a specific function in a protein, peptide or polypeptide.

Fragment: Part of a sequence, either amino acids or nucleotides.

Segment: Part of a sequence, either amino acids or nucleotides.

Sequence: In case of proteins, row arrangement of amino acids attached by peptide bonds; in the case of nucleic acids and/or oligonucleotides, row arrangement of nucleotides attached by phosphodiester bond between the ribose or deoxyribose OH 3′ of a nucleotide and the ribose or deoxyribose phosphate 5′ of the next nucleotide.

Chimeric peptide: Amino acid sequence comprising epitopes of several proteins.

Fusion protein: Amino acid sequence comprising the binding of a fragment or all of a protein to a peptide or other protein of different origin.

Chimeric fusion protein: Amino acid sequence comprising a chimeric peptide or chimeric protein bonded to a fragment or all of a protein.

Carrier: Solid or gel porous matrix where various compounds are embedded and/or adsorbed.

Immunomodulator: Agent that stimulates the immune system.

Vehicle: Solution allowing mixtures of a composition for its use.

Oral vaccine: Formulation and/or composition that prevents diseases enhancing the immune system, which is given orally.

Oral dosage: Quantity of an active ingredient given orally in a timeline and related to the organism weight.

Composition: Formation of a whole or a unified set with a series of elements united in a certain order.

Formulation: It is the process by which different compounds, including the active ingredient, are combined to produce a final drug, including dosage.

Challenge test: Bioassay performed to determine the effectiveness of a treatment.

Region: Similar to the definition of segment, part of a sequence, either amino acids or nucleotides.

### Sequence Description

SEQ ID No. 1: Epitope of a chaperonin HSP60 with amino acid sequence HTKGFAATET.

SEQ ID No. 2: Epitope of a chaperonin HSP60 with amino acid sequence AGGVA.

SEQ ID No. 3: Epitope of a chaperonin HSP60 with amino acid sequence FGSPLITN.

SEQ ID No. 4: Epitope of a chaperonin HSP60 with amino acid sequence KLQE.

SEQ ID No. 5: Epitope of a chaperonin HSP60 with amino acid sequence SVASLILTTE.

SEQ ID No. 6: Epitope of a chaperonin HSP60 with amino acid sequence VTRSALQNAG.

SEQ ID No. 7: Epitope of a pili PI-2a anchor protein with amino acid sequence TYRVIERVSGYAPEYVSFVNGVVTIK.

SEQ ID No. 8: Amino acid sequence of a chimeric peptide of an embodiment of the invention.

SEQ ID No. 9: Amino acid sequence of a SIP protein.

SEQ ID No. 10: Amino acid sequence of a chimeric fusion protein of an embodiment of the invention with SIP.

### DETAILED DESCRIPTION OF THE INVENTION

Taking into account the characteristics mentioned in the background of the invention and under the concept of fusion of specific antigens to immunostimulating proteins, and as a method for obtaining the chimeric peptide of the invention, databases are used to find fish sequences and make a “patchwork” protein; among the databases is The Immune Epitope Database (IEDB, http://tools.iedb.org/main/references). That is, from what is available in the database, the epitopes of the present invention were selected for the realization of the chimeric peptide or protein.

In the present invention epitopes were found in pathogens of teleost fish that have been described as important in the development of cellular and/or humoral immunity. These epitopes are present in different families of pathogens, not necessarily related, but their participation in events related to the immune response, mainly cellular, has been demonstrated. Using these sequences fused to specific antigenic sequences, as a strategy, has the objective of generating an increase in the immune response by both specific and non-specific recognition of them by the immune system, increasing the chances of generating the specific response against the main antigen.

Therefore, the invention of the present application consists in a chimeric peptide named Q in the present application comprises from the N terminal end a residue sequence of amino acid type of at least two equal or different epitopes or domains or fragments in any order, presented by any from SEQ ID No 1 to SEQ ID No 7 or any of the homologous residue sequences of amino acid type from SEQ ID No 1 to SEQ ID No 7, wherein said epitopes are attached among them by an amino acid type linker which can preferably be one or two glycines, alanines or valines Likewise, the invention includes any nucleotide sequence codifying for the chimeric peptide Q or the homologous sequence thereof.

The sequences SEQ ID No 1 to SEQ ID No 6, HTKGFAATET, AGGVA, FGSPLITN, KLQE, SVASLILTTE y VTRSALQNAG respectively, represent conserved domains of 60 KDa chaperonins found in bacteria of Streptococcus genus, Lactococcus y others.

The sequence SEQ ID No 7, TYRVIERVSGYAPEYVSFVNGVVTIK, represents a conserved domain from different bacteria including bacteria of the Streptococcaceae family, belonging to a pili anchor protein.

An embodiment of the chimeric peptide or protein (region Q) of the invention can be the sequence SEQ ID No. 8.

In an additional embodiment of the invention it is provided a chimeric fusion protein named FQ comprising a region named Q (region representing an artificial chimeric peptide that enhances the immune response) attached to another region called F (region representing a fusion peptide or fusion protein), wherein the region F is in the N terminal end and it is a protein or fragment known for stimulating the immune system in fish, the invention includes any nucleotide sequence codifying for the chimeric fusion protein FQ or the homologous sequence thereof.

The region F of the chimeric fusion protein can be a protein or fragments thereof, homologous sequences of amino acid type or fragments thereof corresponding to a Modified Surface immunogenic protein (SIP) with sequence SEQ ID No 9 Likewise it has to be included any homologous sequence to proteins related to SEQ ID No 9.

And therefore it can be obtained from SEQ ID No 9 an embodiment of the chimeric fusion protein FQ represented by SEQ ID No. 10.

The generation or formation of chimeric fusion proteins and when the fragments or domains or epitopes that make them up are required, is carried out “in vitro”, that is, it is only required to establish the desired sequence by bioinformatics and the amino acid sequence is made by non-biological synthesis, which is then purified.

Likewise, if any DNA sequence is required that codifies for the chimeric fusion protein of the invention or the fragments or domains or epitopes that make it up, it can be cloned into any expression vector to generate the protein of interest using the corresponding restriction enzymes, or recombination sequences; those vectors can be for expression in E. coli by induction with IPTG, as well as for expression in other bacteria, fungi, animal or plant cells, or expression systems involving viruses.

The culture of the type of cell for the intracellular or extracellular production of the chimeric fusion protein of the present invention can be carried out in any of the ways known to the person skilled in the art, it could be batch culture, fed batch, continuous and also in any of the equipment available for this purpose, considering the nutritional and energy requirements related to the source of carbon, nitrogen, micro and macroelements as the case may be, as well as cell growth conditions such as aeration, agitation, temperature and pH. The purification of the chimeric fusion protein is also carried out according to the expression system used and considers, depending on the case, mechanical and/or chemical cell disruption, centrifugation and/or filtration, microfiltration, ultrafiltration, diafiltration, precipitation with ammonium sulfate, dialysis, size exclusion chromatography, ion exchange, affinity, immunoseparation, lyophilization, drying and crystallization.

Said chimeric fusion protein after being obtained is part of a composition that presents immunomodulators between 0.1 and 10% such as palm oil or other natural oils of vegetable origin, 3-glucan from yeast or from another source, aluminum hydroxide, potassium aluminum phosphate, CpG microbial components, bacterial lipopolysaccharides LPS, tetanus toxin and measles virus, Mycobacterium butyricum, Mycobacterium bovis, Mycobacterium chelonae, mycobacterial cell wall, flagellin, interleukins, chemokines, vitamin C, vitamin E, saponins and/or mixtures thereof. The immunomodulators alone and/or combinations thereof are supported in a carrier of chitosan, alginate, liposomes, biodegradable microspheres, PLGA nanoparticles.

The preparation of the composition in the carrier is carried out in the way that the technician versed in the matter knows, which basically is to generate the crosslinking or entrapment in the web or capsule considered as carrier in the presence of the chimeric fusion protein and immunomodulators, allowing adequate time for the composition to stabilize and then it is performed a drying and/or lyophilization. In some cases, some immunomodulators must be added after having the carrier stabilized with the chimeric fusion protein.

La composition is supplied to the fish orally using a solution between 0.1 and 10% of carboxymethylcellulose, stabilizers, colorants, ionic and non-ionic surfactants, alone and/or combinations thereof, as vehicle for dosing alone or in a mixture with food, in order to avoid additional handling of fish that would lead to episodes of stress. The dosage of the chimeric fusion protein ranges from 1 to 1000 μg/dose, regardless of whether it is mixed with food or not prior to ingestion. Generally, it is a mixture that is made between the composition and the vehicle for the dosage.

The use of the composition and/or formulation as vaccine is focused on the prevention of diseases caused by bacteria in fish breeding.

The invention is not limited to the above embodiments of chimeric fusion proteins, but also extends to compositions, formulations and vaccines containing any of said chimeric fusion proteins set forth in the present invention.

Although the present invention has been described with preferred embodiments, it is understood that modifications and variations that retain the spirit and object of this invention are within the scope of the subject matter to be claimed.

### EXAMPLES

### Example 1

SIP-FUSION (49 KDa) Expression in E. coli BL21 (DE3)

The SIP-FUSION expression was performed in E. coli BL21 (DE3), which was previously transformed with the corresponding plasmid pRSETA SIP-FUSION. It was made a starter overnight culture in 30 ml of Luria Bertani (LB) Broth with 50 mg/ml of ampicillin. The starter culture was used to inoculate 300 ml of LB with 50 mg/ml ampicillin, which were incubated overnight at 37° C. with 250 rpm of stifling. The induction of the expression was made to an O.D. (optical density) of 0.6 with 0.1 mM of IPTG. It was kept 1 ml of culture for the quantitative analysis.

### Example 2

Obtaining of the SIP-FUSION Lysate Expressed in E. coli BL21 (DE3)

It was performed a starter overnight culture in 30 ml of Luria Bertani (LB) Broth with 50 mg/ml of ampicillin. The starter culture was used to inoculate 300 ml of LB with 50 mg/ml ampicillin, which were incubated overnight at 37° C. with 250 rpm of stifling. The broth was centrifuged at 3000 rpm and after that the bacteria were resuspended in 30 ml of buffer PBS. The lysis was made using a tip of 13 mm in 5 cycles of 30 seconds each one (amplitude 50%) on an ice bath. Subsequently the lysate was preserved at −80° C.

### Example 3

SDS PAGE/Western Blot Analysis

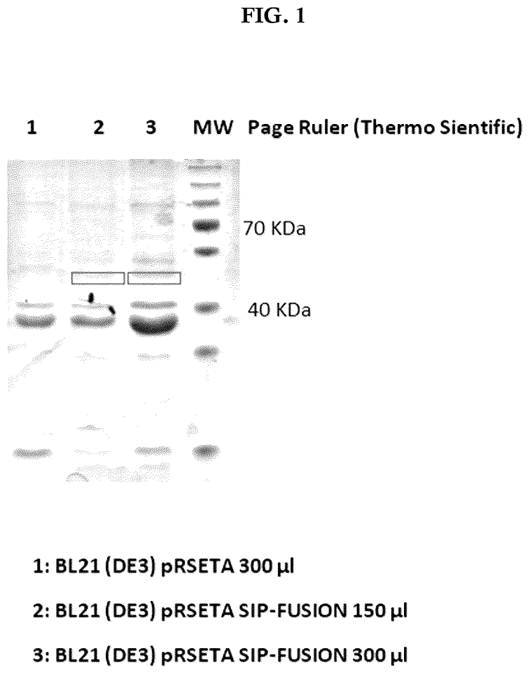

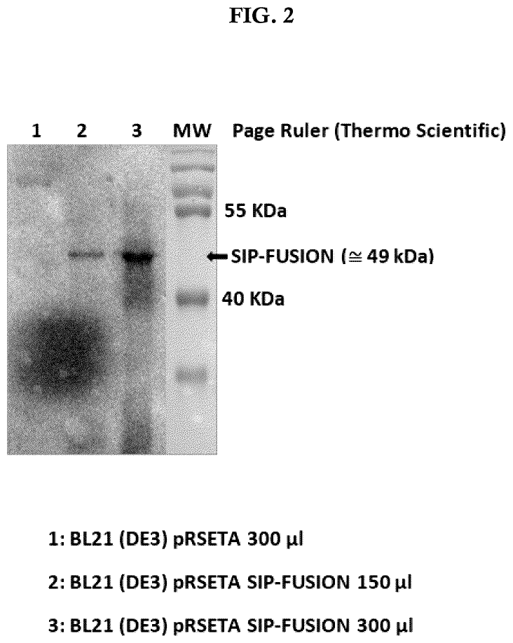

It was analyzed 300 and 150 μl of the lysate, which were centrifuged at 13000 rpm and subsequently resuspended in running buffer (Laemmli sample buffer, 4% SDS, 20% glycerol, 10% 2-mercaptoethanol, 0.004% bromophenol blue and 0.125 M Tris HCl, pH approx. 6.8). As control, it was used a 300 μl lysate of an E. coli BL21 (DE3) culture previously transformed with the empty plasmid. El SDS-PAGE y Western Blot, were made according to the standard protocol (N Am J Med Sci. 2012 September; 4(9): 429-434. doi: 10.4103/1947-2714.100998) using 6×-His Tag Monoclonal Antibody (HIS.H8), HRP (Thermofisher) as detector antibody. The FIGS. 1 and 2 show in the SDS-PAGE gel and in the Western Blot respectively the presence of the SIP-FUSION expressed protein.

The SDS-PAGE/Coomasie Blue analysis (FIG. 1) evidence the presence of a band (inside a rectangle) with the expected molecular weight of SIP-FUSION protein, which is not seen in the control related to transformed bacteria with the empty plasmid. The Western Blot (FIG. 2) analysis using an antibody against the 6×HIS tag in the peptide indicates that the mentioned band corresponds to a SIP-FUSION protein, due to this is the only protein produced by the cell containing the 6×HIS tag.

### Example 4

Enhancing the humoral immune response due to the presence of the portion FUSION (region Q) of the SIP-FUSION (FQ).

Objective: Verify the FUSION capability for increase the humoral immune response of SIP peptide.

For this experiment it was made 3 formulations:

Formulation 1 (F1): SIP-FUSION lysate expressed in E. coli BL21 (DE3)+incomplete Freund adjuvant. Dose: 200 μg of SIP-FUSION.

Formulation 2 (F2): SIP lysate expressed in E. coli BL21 (DE3)+incomplete Freund adjuvant. Dose: 200 μg of SIP-FUSION.

Formulation 3 (F3): Saline Solution+incomplete Freund adjuvant.

It was used 15 individuals of Oreochromis niloticus of 50±5 g weight, which were kept at constant temperature 25±2° C., dissolved oxygen 8-10 mg/L, Ammonium: 0-1 mg/L, Nitrites: 0-1 ppm, Nitrates: 0-40 ppm, pH: 7-8.5 and fed ad libitum.

For each condition (F1, F2 y F3) (n=5) the fish were immunized with con 100 uL of the corresponding formulation administered by intraperitoneal (IP) route, 5 fish previously anesthetized. The immunization scheme was of two IP doses, at day 0 and at day 21.

Peripheral blood samples were taken from the caudal vein and the antibody titer against Streptococcus agalactiae was performed according to the protocol “Microtiter agglutination test” in a 96-well plate U bottom.

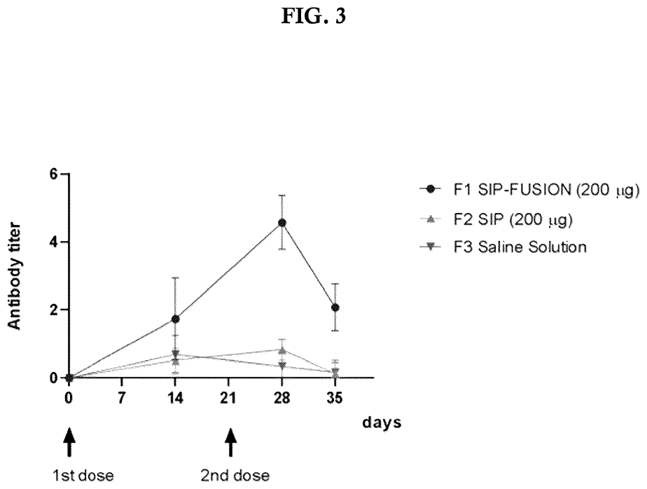

The FIG. 3 shows that the antibody titer after two doses of SIP-FUSION (FQ) is significantly higher (p<0,0001) than the antibody titer of the SIP, which lacks the immunostimulant FUSION portion (region Q). It is demonstrated in this manner that not only SIP has the capability to generate humoral immune response able to recognize and agglutinate the Streptococcus bacteria, but also it is seen the enhancing effect of the FUSION portion (region Q) built of immunostimulating sequences of amino acids.

### Example 5

Protection effectiveness against the challenge with Streptococcus agalactiae (UEL12) with the oral administration of SIP-FUSION (FQ) in particles of alginate-chitosan.

Objective: to evaluate the protection against the challenge with Streptococcus agalactiae (UEL12) conferred with immunization with SIP (region F) or SIP-Fusion (FQ), included with particles of alginate-chitosan as vehicle and 1-6 β-glucans as adjuvant.

For this experiment it were prepared 3 oral formulations:

Formulation 1 (F1): particles of alginate-chitosan containing: 1-6 β-glucans+SIP-FUSION. Dose: 200 μg de SIP-FUSION

Formulation 2 (F2): particles of alginate-chitosan containing: 1-6 β-glucans+SIP. Dose: 200 μg de SIP.

Formulation 3 (F3): particles of alginate-chitosan containing: 1-6 β-glucans.

For the formation of the particles of alginate-chitosan 10 ml of a CaCl₂) solution (3.35 mg/ml) (with or without antigen) were added drop by drop to a 30 ml of an alginate solution (3 mg/ml, 1% p/v 1-6 β-glucans, pH 5.1), in which the antigen was previously dissolved with 1000 rpm of stifling during 30 minutes. Then 20 ml of the chitosan solution were added (0.8 mg/ml, 1% v/v acetic acid) and incubated with stirring at 4° C. over night for stabilization. After that, the nanoparticles were separated by centrifugation at 1200 rpm (Li et al., 2008).

The oral immunization scheme consisted in the administration of the doses of oral composition (200 μg of antigen/dose) divided in 5 fractions of 40 μg each one, which are supplied one per day during 5 days in a mix with an amount of food equal to a 2% of the animal's weight per fraction. After 21 days the treatment is repeated, completing in this way two doses of 200 μg of antigen per fish.

It was used 45 individuals of Oreochromis niloticus with 50±5 g weight, which were kept at constant temperature 25±2° C., dissolved oxygen 8-10 mg/L, Ammonium: 0-1 mg/L, Nitrites: 0-1 ppm, Nitrates: 0-40 ppm, pH: 7-8.5 and fed with 2% of the fish weight/day.

It was executed the oral immunization scheme previously described and 15 fish per condition received the treatment (F1, F2 y F3) (n=15), for a total of two doses of 200 μg with the corresponding antigen (F1 y F2) or empty particles (F3) per fish, respectively.

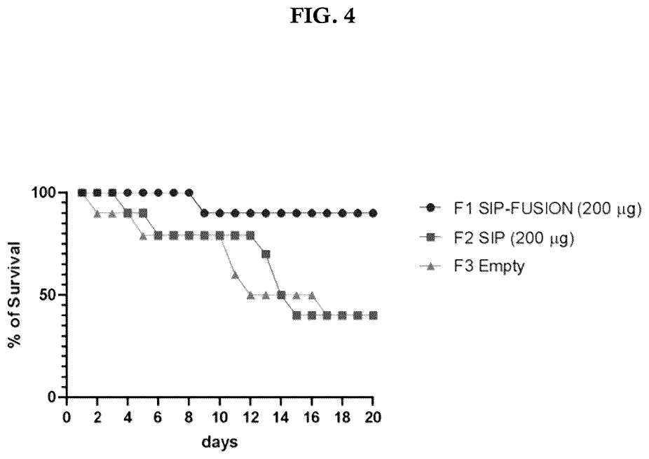

At day 28 post-inmunization, the fish were exposed in the challenge test against Streptococcus agalactiae (UEL12) following the methodology described in Pretto-Giordano et al. (2010). It is counted the dead fish every day during 20 days and it is calculated the absolute survival percent (FIG. 4).

The FIG. 4 shows the oral immunization with the particles of alginate-chitosan containing SIP-FUSION (FQ) and 1-6 β-glucans as adjuvant, which protected in a 90% against the challenge with the pathogen, being this a significantly superior (50%, p<0,0001) protection compared to the treatments with SIP (region F) (40%) or empty particles (40%). It is demonstrated in this manner the enhancing effect of the immunostimulating FUSION portion (region Q) of the SIP-FUSION in the protection against the challenge with Streptococcus agalactiae (UEL12).

It should be clearly understood that the invention defined by the included claims is not limited to the specific embodiments indicated in the foregoing description, but encompasses variants that do not depart from the scope or spirit of the present invention.

Therefore, what is claimed in the invention of the present application is:

1. An artificial chimeric peptide Q comprising
a residue sequence of amino acid type of at least two equal or different epitopes or domains or fragments represented by any of sequences SEQ ID No 1 to SEQ ID No 7 or any of the homologous residue sequences of amino acid type from SEQ ID No 1 to SEQ ID No 7, wherein said epitopes are attached among them by an amino acid type linker.

2. The artificial chimeric peptide Q according to the claim 1, wherein the residue sequence of amino acid type comprises any of sequences SEQ ID No 1 to SEQ ID No 7, or any of the homologous residue sequences of amino acid type from SEQ ID No 1 to SEQ ID No 7, in any order.

3. The artificial chimeric peptide Q according to the claim 1, wherein el linker joining any of two fragment or domains or epitopes comprises preferably one or two glycines, alanines or valines.

4. The artificial chimeric peptide Q according to the claim 1, wherein the residue sequence of amino acid type comprises the sequence SEQ ID No 8, or any homologous sequence to SEQ ID No 8.

5. A chimeric fusion protein FQ comprising a region named Q corresponding to the artificial chimeric peptide Q according to the claim 1, joined to another region named F, wherein the region F consists in any domain or fragment or region or epitope of a protein or a full protein known for stimulating the immune system in fish.

6. The chimeric fusion protein FQ according to the claim 5, wherein the region F comprises any domain or fragment or region or epitope of a protein or a full protein of the modified surface immunogenic protein (SIP) with sequence SEQ ID No 9, also including any homologous sequence to said previous sequence SEQ ID No 9.

7. The chimeric fusion protein FQ according to the claim 5, wherein the residue sequence of amino acid type comprises the sequence SEQ ID No 10, or any homologous sequence to SEQ ID No 10.

8. The chimeric fusion protein FQ according to the claim 5, wherein said protein FQ comprises being part of a vaccine or in a mixture with food to stimulate the immune system.

9. A composition comprising: a) One or several immunomodulators and/or combinations thereof, in a mix with a chimeric fusion protein FQ according to the claim 5, supported on a carrier, and
b) A solution as vehicle.

10. The composition according to claim 9, wherein the immunomodulator or immunomodulators comprises among any of palm oil or other natural oils of vegetable origin, (3-glucan from yeast or from another source, aluminum hydroxide, potassium aluminum phosphate, CpG microbial components, bacterial lipopolysaccharides LPS, tetanus toxin and measles virus, Mycobacterium butyricum, Mycobacterium bovis, Mycobacterium chelonae, mycobacterial cell wall, flagellin, interleukins, chemokines, vitamin C, vitamin E, saponins and/or mixtures thereof, whose amount in the composition is between 0.1 and 10%.

11. The composition according to claim 9, wherein the immunomodulators consisting of palm oil and β-glucan.

12. The composition according to claim 9, wherein the carrier comprises among any of chitosan, alginate, liposomes, biodegradable microspheres, PLGA nanoparticles, or a mixture thereof.

13. The composition according to claim 9, wherein the carrier consisting of alginate with chitosan.

14. The composition according to claim 9, wherein the carrier comprises a solution as vehicle comprising among any of carboxymethylcellulose, stabilizers, colorants, ionic and non-ionic surfactants, alone and/or combinations thereof, whose amount in the composition is between 0.1 and 10%.

15. The composition according to claim 9, wherein the solution as vehicle consisting of carboxymethylcellulose.

16. The composition according to claim 9, wherein said composition comprises being dosified with the chimeric fusion protein FQ in the range between 1 a 1000 μg/dose, as a vaccine or in a mixture with food for stimulating the immune system.

17. A method for preparation of a composition comprising:
a) Find in databases epitopes of pathogens of teleost fish that have been described as important in the development of cellular and/or humoral immunity.
b) Join these sequences to obtain the chimeric peptide Q and/or fuse them with specific antigenic sequences to obtain the chimeric fusion protein FQ. Wherein the chimeric peptide Q is a residue sequence of amino acid type of at least two equal or different epitopes or domains or fragments represented by any of sequences SEQ ID No 1 to SEQ ID No 7 or any of the homologous residue sequences of amino acid type from SEQ ID No 1 to SEQ ID No 7, and the chimeric fusion protein FQ is the chimeric peptide Q joined to other region named F, wherein the region F comprises any domain or fragment or region or epitope of protein or full protein known for stimulating the immune system in fish.
Wherein said epitopes are attached among them by an amino acid type linker, being preferably one or two glycines, alanines or valines.
c) Perform the non-biological synthesis or biological synthesis of a chimeric peptide Q and/or a chimeric fusion protein FQ.
d) Purify the chimeric peptide Q and/or the chimeric fusion protein FQ.
e) Mix immunomodulators between from 0.1 to 10% with a carrier of chitosan, alginate, liposomes, biodegradable microspheres, PLGA nanoparticles, alone and/or combinations thereof.
f) Generate the crosslinking or entrapment in the web or capsule considered as carrier in the presence of the chimeric peptide Q and/or the chimeric fusion protein FQ and immunomodulators, allowing adequate time for the composition to stabilize and then it is performed a drying and/or lyophilization. In some cases, some immunomodulators must be added after having the carrier stabilized with the chimeric fusion protein FQ.
g) Mix when is required the carrier supporting immunomodulators and the chimeric peptide Q and/or the chimeric fusion protein FQ with a solution between from 0.1 to 10% of carboxymethylcellulose, stabilizers, colorants, ionic and non-ionic surfactants, alone and/or combinations thereof.

18. The method according to claim 17, wherein the immunomodulators are selected among any of palm oil or other natural oils of vegetable origin, β-glucan from yeast or from another source, aluminum hydroxide, potassium aluminum phosphate, CpG microbial components, bacterial lipopolysaccharides LPS, tetanus toxin and measles virus, Mycobacterium butyricum, Mycobacterium bovis, Mycobacterium chelonae, mycobacterial cell wall, flagellin, interleukins, chemokines, vitamin C, vitamin E, saponins and/or mixtures thereof.

19. The method according to claim 17, wherein in order to obtain the peptide or protein through biological synthesis it is required before mixing with the immunomodulators and carrier
c.1) Cloning the ADN sequence codifying for the chimeric peptide Q and/or the chimeric fusion protein FQ into any expression vector to generate the protein of interest using the corresponding restriction enzymes, or recombination sequences; those vectors can be for expression in E. coli by induction with IPTG, as well as for expression in other bacteria, fungi, animal or plant cells, or expression systems involving viruses
c.2) Culture the type of cell for the intracellular or extracellular production of the chimeric fusion protein of the present invention that can be carried out in any of the ways known to the person skilled in the art, it could be batch culture, fed batch, continuous and also in any of the equipment available for this purpose, considering the nutritional and energy requirements related to the source of carbon, nitrogen, micro and macroelements as the case may be, as well as cell growth conditions such as aeration, agitation, temperature and pH.
d.1) Purify the chimeric peptide Q and/or the chimeric fusion protein FQ according to the expression system used and considers, depending on the case, mechanical and/or chemical cell disruption, centrifugation and/or filtration, microfiltration, ultrafiltration, diafiltration, precipitation with ammonium sulfate, dialysis, size exclusion chromatography, ion exchange, affinity, immunoseparation, lyophilization, drying and crystallization.

20. The method according to claim 17, wherein the region F comprises any domain or fragment or region or epitope of a protein or a full protein of the modified surface immunogenic protein (SIP) with sequence SEQ ID No 9, also including any homologous sequence to said previous sequence SEQ ID No 9.
